(19) United States
(12) Patent Application Publication
(10) Pub. No.: US 2020/0198971 A1
(43) Pub. Date: Jun. 25, 2020

(54) Method for chemical modification of fluorinated carbons with sulfur-containing substance

(71) Applicants: Alexander ZADERKO, Brovary City (UA); Vasyl PRUSOV, Kyiv City (UA); Vitaliy DIYUK, Kyiv City (UA)

(72) Inventors: Alexander ZADERKO, Brovary City (UA); Vasyl PRUSOV, Kyiv City (UA); Vitaliy DIYUK, Kyiv City (UA)

(21) Appl. No.: 16/500,560

(22) PCT Filed: Apr. 18, 2017
(86) PCT No.: PCT/UA2017/000047

§ 371 Date: Oct. 3, 2019

(51) Int. Cl. C01B 32/05; C01B 32/354; C01B 32/168; C09C 1/56; C07C 309/20; D01F 9/12; D01F 11/16

(52) U.S. Cl. CPC C01B 32/05; C01B 32/354; C01B 32/168; C09C 1/56; C01P 2002/82; D01F 9/12; D01F 11/16; C01P 2002/85; C01P 2002/88; C07C 309/20

(57) ABSTRACT

A method of sulfurization of fluorine-containing carbon materials obtained by heating of carbon materials in contact with fluorocarbons or fluorine-containing derivatives thereof. Claimed method allows obtaining a wide range of fluorine-containing carbon materials with grafted sulfur functionalities. Claimed materials can be used in industry as novel acid-base catalysts with high stability in any aggressive medium. Another embodiment of the invention can be used for producing electrodes of metal-sulfide batteries or as a specific sorbent, metals or nanoparticles support.

This invention expands, clarifies and complements that disclosed in UA patent application for the invention a 2016 00510 and based on this application. The invention further relates to the chemical industry and provides the chemical modification method of surface of fluorinated porous and/or highly dispersed carbon precursors, which are produced by the method claimed in the patent UA 110301 C2, Pub. Date: Dec. 10, 2015; Intern. Pub. WO 2016072959, Pub. Date: May 12, 2016; and UA patent application for the invention a 2015 11416, Pub. Date: Mar. 10, 2016. The method discloses the subsequent stages of the modification of such materials with sulfur-containing substances (sulfurization) and further stages in order to obtain fluorine-containing carbon materials, which surface layer is enriched with sulfur functionalities, including highly acidic groups, e.g. —CF₂-SO₃H.

Above-mentioned claimed methods are dedicated for obtaining the fluorine-containing carbons, which structure may also include fragments with unsaturated CC bonds and/or containing halogen apart from fluorine. The result of the modification according to these methods is the carbon material with grafted fluorine-containing functional groups. For example, the carbon material treated with Freon R-12 (CF₂Cl₂) contains chlorine and fluorine, while treated with Freon R-114B2 (BrCF₂CF₂Br, Halothane®) fluorine and bromine, respectively, both of obtained carbon materials have a reactive multiple CC bonds in the structure of the carbon matrix. After the treatment with Freon R-134a, Forane® (H₂FC-CF₃), we believe that the carbon material contains active unsaturated CC bonds in fragments of fluorinating agents that grafted to the surface. Overall, we—the authors, according to international practice, assigned a general name—Fluocar® to the fluorine-containing materials obtained as described in these patent publications.

The chemical properties of Fluocar®, obtained by the modification in liquid or gas phase, will vary slightly, and this difference has an insignificant effect on subsequent addition of sulfur. Halogens, multiple carbon bonds, vacancies and/or carbon matrix modifiers can serve as centers for the addition of sulfur-containing substances. Also, the grafted modifier or fluorine-containing carbon matrix may contain oxygen, which can also serve as a center for the sulfur addition, since this oxygen during the thermal transformations of the carbon surface layer is capable of forming defects inside the graphene-like matrix of the carbon material.

The goal of this work was to develop a method for further modification of Fluocar® material to create sulfur-containing solids, as a variant with super acidic properties. Direct sulfonation e.g. —H atom replacement, replacement of active halogen, typically, Br or Cl, with sulfur-containing group or sulfur addition to C═C double bond is a well-known method for the preparation of sulfur compounds in organic synthesis. Furthermore, the treatment with sulfur-containing compounds is the classical route to functionalize by sulfur a wide range of carbon materials [1-3]. Depending on the synthesis condition, nature of sulfur-containing reagent, the resulted modified carbon material can have the grafted groups of different nature: mercapto-, sulfide, di- or polysulfide-, sulfone or sulfonic groups, etc.

Fluorine-containing carbon material can be obtained by processing with fluorocarbon or derivative from wide range of initial carbon materials, it can be chosen among activated carbon, coke, pitch coke, charcoal or carbon fibers from synthetic or natural row, carbon nanotubes, carbon black, graphene, carbonizates, nanodiamonds, fullerenes or fullerite, or other suitable carbon.

The most interesting materials, in our opinion, are those containing sulfonic groups grafted to the surface through difluoromethylene or other fluoralkylene groups because these centers are characterized by extremely high acidity (pKₐ<<0, alike the acidity of perfluoroalkyl sulfonic acids) [4]. Some of the valuable properties of this materials, e.g. relatively high thermal and superior chemical resistance of surface functionalities, can be used for the production of fuel cells components [5], acid catalysts [6-9], membrane electrodes [10].

The technical result of this work is development of the chemical modification method for the fluorine functionalized carbons, which are obtained by chemical interaction between porous and/or highly dispersed carbon and fluorocarbon or its derivative at the heating, by introducing them in contact with sulfur-containing substance in the reaction medium. During the reaction process, the sulfur-containing groups substitute active halogen or —H atom, or join to the active multiple C—C bonds of carbon matrix and/or fluorinating agent grafted residues.

Moreover, these multiple C—C bonds or unsaturated valences (defects) of carbon matrix can be formed during thermal transformation of oxygen groups of fluorinated reactant residues or as a source of oxygen can act the oxygen containing groups of the carbon surface. Subsequently, the S-containing carbon material may be subjected to hydrolysis and/or oxidation of the sulfur derived groups giving resulting material.

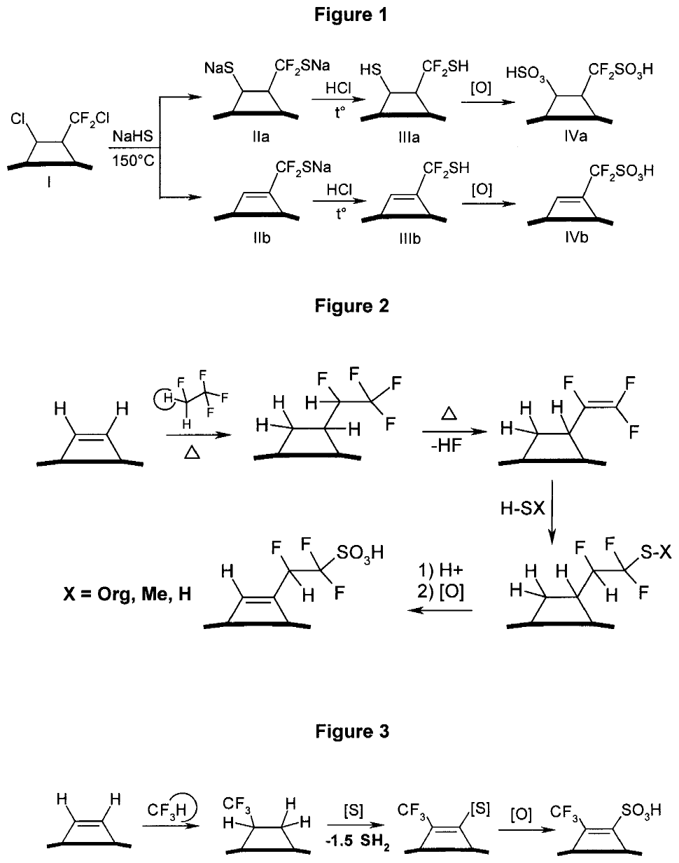

For example, the chemical modification of Fluocar® material, which was obtained by the chemical modification of the carbon material with Freon R-12, with sulfur containing substance, can be explained by schema (FIG. 1).

FIG. 1. Modification of chlorine-containing fluorinated carbon material with sodium hydrosulfide, followed by hydrolysis and oxidation.

In the first stage, the fluorine-containing precursor obtained by the treatment of carbon material with R-12 Freon (surface structure I) reacts with sodium hydrosulfide at a temperature of 150° C. to form S-adduct in the sodium salt form (surface mercapto group IIa). It is also possible the partial elimination of HCl in the alkaline environment with the renewal of the double bond C═C (IIb). In this case, the sodium hydrosulfide also plays the role of a base. In the second stage, the S-derivative was subjected to hydrolysis by treatment with diluted hydrochloric acid to form a fluorine-containing carbon containing HS-groups (Ma and Mb). In turn, thiol groups under the action of oxidants conversed into HSO₃-groups, i.e., products (IVa) and (IVb).

The chemical modification of carbon material with Freon R-134a and its further sulfurization, in the presence of a base, can be illustrated as follows (FIG. 2).

FIG. 2. Modifying of fluorinated carbon material that containing unsaturated C—C bonds with a derivative of hydrogen sulfide, followed by hydrolysis and oxidation of sulfur to sulfonic groups.

When carbon material treated by Freon R-23, fluoroform (Fluoryl®, CHF₃), its modification with sulfur-containing substance (e.g. elemental sulfur, polysulfide or CS₂) at heating and next stages can be explained as follows:

FIG. 3. Modification of carbon material fluorinated by Fluoryl® that contain active hydrogen with sulfur compounds, and imagining subsequent stages of hydrolysis and oxidation of S-adduct to surface sulfonic groups.

The resulting sulfonated fluorinated carbon materials were subjected to chemical analysis for sulfur and halogens, analyzed by XPS, N₂ adsorbtion, coulometric titration and through thermogravimetric method and TPD-mass and TPD-IR spectrometry. It was found that the sulfur contained in such samples is assigned to sulfonic groups (from TPD-mass and TPD-IR data on SO₂ evolution), and, these groups desorption passes at relatively high temperatures, in 120-550° C. temperature range. Thus, we obtained sulfur-containing materials having a thermal stability superior to those that do not contain fluorine. Its thermal stability is similar to Nafion® H resin.

According to catalytic tests in gas phase alcohol dehydration reaction conducted in the presence of catalytic materials, was found that modified fluorinated carbons that has HSO₃-functionalities show the higher conversion and excellent thermal stability under reaction conditions, better than conventional sulfonated carbons, and are close to the relevant characteristics of Nafion® H resin.

The same technical result can be achieved through a variety of options for synthesis: instead of sodium hydrosulfide it can be applied sulfide or polysulfide of alkaline or other metal soluble salts [10], its derivatives, including mercaptoacetic acid (or its salts) [11, 12], thiourea or its pyrolysis products [13], phosphorous sulfide [14] or other suitable sulfurizing agent; active hydrogen, halogen or multiple C—C bonds or unsaturated carbon fragments can interact with sulfide, sulfite, thiosulfate, metabisulfite, dithionite or other salts of sulfurous acids (salts) or sulfurous anhydride [15], organic sulfides or disulfides, chlorosulfonic acid, elemental sulfur, in gaseous or liquid medium; hydrolysis can be carried out apart of hydrochloric acid with other acid, alkali, or even water steam; thiol groups oxidation can be carried with an assistance of substances such as hydrogen peroxide, nitrous acid, chlorine oxides, hypochlorite, chlorate, chromate, permanganate ions or peroxo salts, or through nitrogen oxides, ozone or oxygen in an alkaline, neutral or acidic medium. Thus, the proposed method for obtaining the fluorinated carbon materials with sulfur-containing functional groups can be implemented by three stages: addition of sulfur-containing substances (S-adduct formation), S-adduct hydrolysis, and followed oxidation. In a case of some sulfurizing reagents (e.g. chlorosulfonic acid, SO₂ or SO₃), HSO₃-group can be obtained directly in the one stage. This made hydrolysis and oxidation stages unnecessary.

Additionally, product of sulfurization stage of fluorine-containing carbon material can be usable in some industrial applications, for example, as an electrode for metal-sulfur batteries, analogous to [16], or as specific sorbent, metal or nanoparticles support material.

For materials, obtained by method claimed, authors agreed to use name Fluocar®.

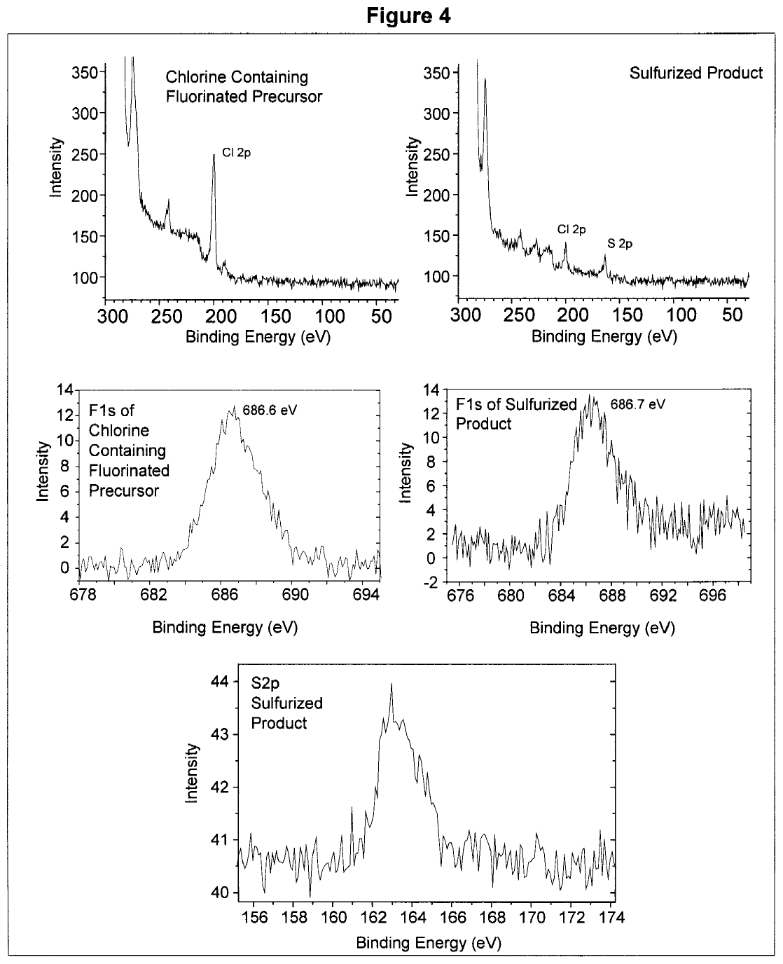

FIG. 4. XPS Spectra of fluorinated carbon material: SCN treated with R-12 Freon and then sulfurized by NaHS product in region of 300-60 eV; F 1s XPS spectra of them and S 2p spectra of sulfurized product

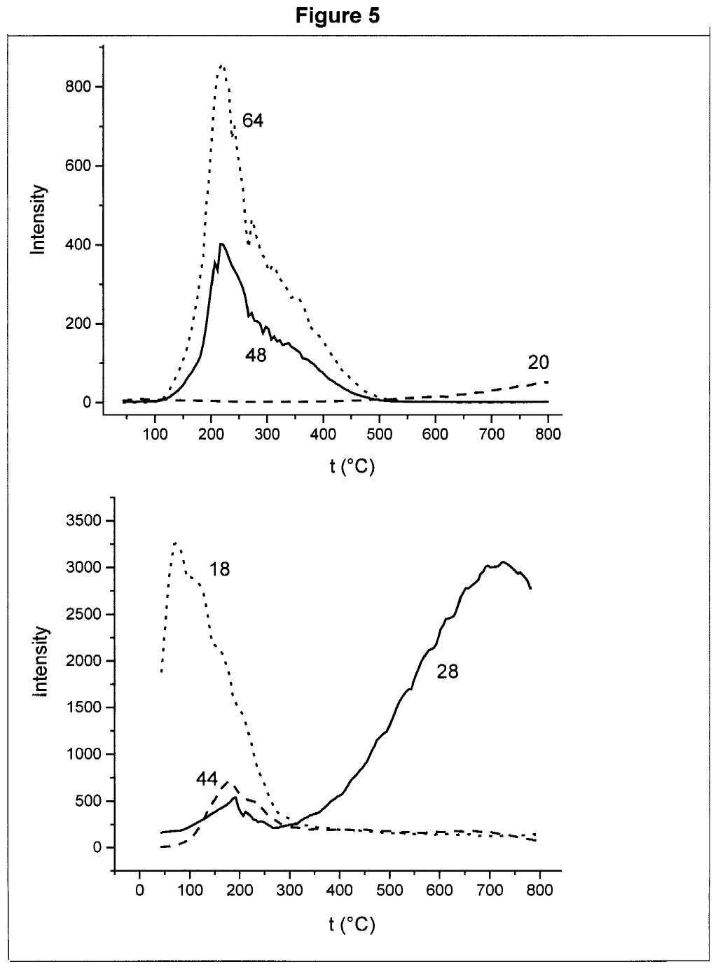

FIG. 5. TPD-mass spectra of SCN treated with R-12 Freon, sulfurized with NaHS and oxidized end product for m/z=18, 20, 28, 44 and 64 fragments

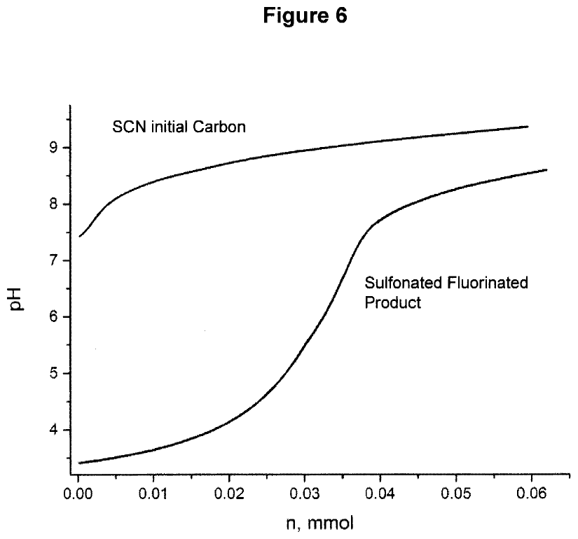

FIG. 6. Coulometric acid-base titration curves for initial SCN active carbon and treated with R-12 Freon, sulfurized with NaHS and oxidized end product

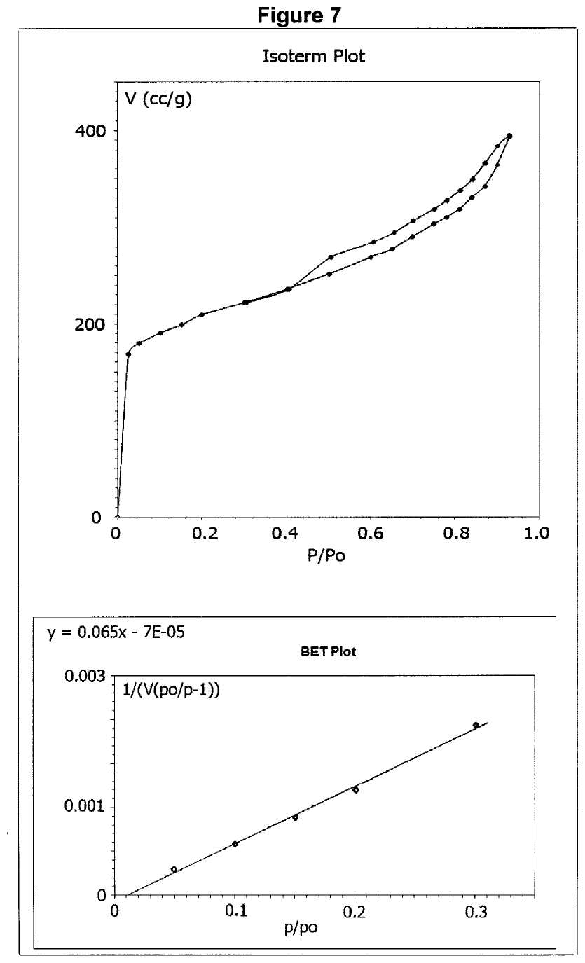

FIG. 7. N₂ adsorption isotherm and BET Plot for KAU active carbon, that was chemically modified with Freon R-13, then sulfurized with H₂S and then oxidized

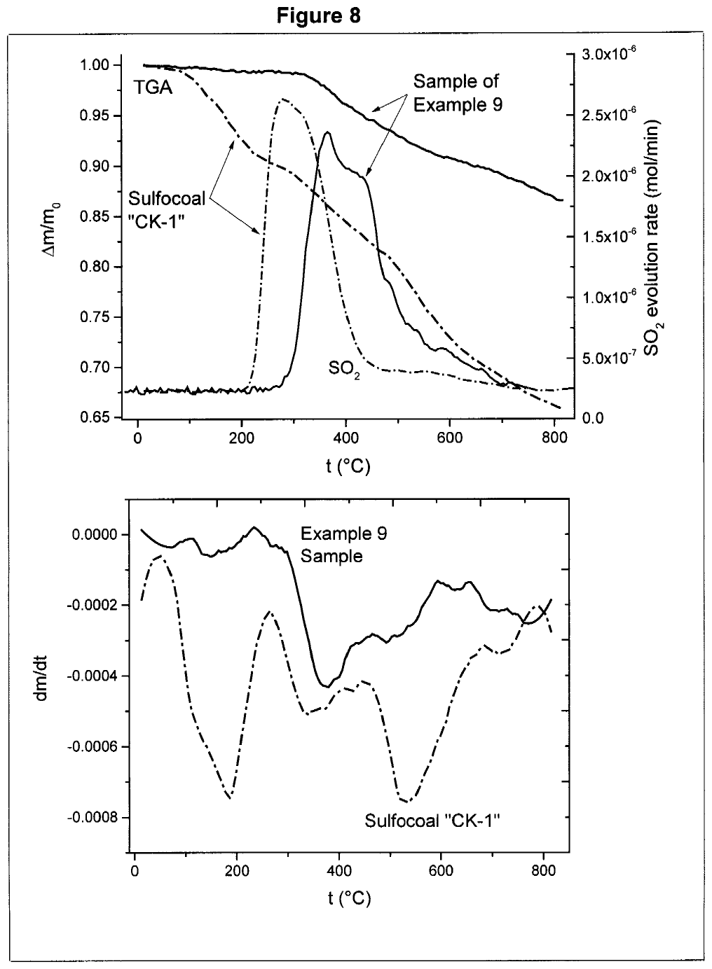

FIG. 8. TPD-IR curves of SO₂-evolution, TGA/DTG of Norit® 830 W active carbon sample, that was modified with R-125 Freon, sulfurized with elemental sulfur and then oxidized, in comparison with washed with Na₂CO₃/HCl commercial sulfonated coal

The invention is illustrated by the following Examples:
1. 3 g of fluorinated carbon material (fluorine content is of 1.72 mmol/g, chlorine is of 2.02 mmol/g), which was obtained by reacting when heated to 560° C. of Freon R-12 (Difluorodichloromethane, CF₂Cl₂) with “SCN” active carbon (raw material—vinylpyridine resin) was introduced in a contact with 3 g of trihydrate of sodium hydrosulfide (NaHS·3H₂O, pur.) in a Teflon beaker in a hermetic glass autoclave at 145° C. for 12 hours. After the reaction product was washed with water, 5% hydrochloric acid, again with water and treated with 50 ml of 1.00 g of Berthollet salt in 2M nitric acid for 18 hours. The resulting product is separated from the solution, washed with water, poured into 12 hours in 3% sodium carbonate solution to neutralize acids and remove adsorbed sulfates, thoroughly rinsed with water and treated with 5% hydrochloric acid to restore acid state of sulfonic groups. After treatment with acid, material was washed with water to pH 4.5. The resulting carbon material according to the results of chemical analysis contains 1.65 mmol/g of fluorine, 0.62 mmol/g of chlorine and 0.45 mmol/g of sulfur. Unlike the original fluorinated material having a XPS spectrum clear but slightly asymmetric signal of fluorine (at 686.6 eV) and chlorine (doublet 199.5 eV, 201.0 eV) derivative after the stage of hydrolysis has about a half strength signal of chlorine (at 199.7, 201.4 eV) at nearly constant strength of fluorine more asymmetric signal (in 686.7 eV), and complex signal of sulfur in mercapto-form (with 162.8 eV and 164.0 eV complex components) (FIG. 4). By TPD-mass (FIG. 5) method it was shown that final product desorb sulfur in SO₂ form (m/z 64 and 48) in a temperature range of 125-475° C. with maximums at 220 and 350° C. Signal with m/z 32 is negligible in all investigated temperature range, that confirm absence of elemental sulfur in sample. In the temperature range of sulfonic groups decomposition also observed evolving of CO₂ and H₂O (m/z 44 and 18 respectively). Intensive CO evolution (m/z 28) observed above 320° C. with maximum at 730° C. and can be assigned to carbonyl or ether groups [17]. Water forms, desorbed at the temperature exceeded 120° C. can be assigned to hydrate forms of HSO₃-functionalities. Evolution of HF (m/z=18) is observed at a temperature higher than 520° C. and occurs in a small quantity. Catalytic study in the gas phase reaction of i-propanol dehydration give a temperature of 100% conversion is 115° C. for this sample (250 mg), showing an excellent stability in reaction media in cyclic regime. By coulometric titration (FIG. 6) it was shown the presence of strong acid function on a surface of a sample (0.45 mmol/g). According to the final sample properties established by methods of TPD-IR, mass spectrometry, catalytic tests and coulometric titration it's can be concluded that sulfur contained in the sample as a grafted sulphonic acid state.
2. Synthesis was conducted similar to that described in Example 1, but as a starting carbon material was taken Norit® 830W active carbon, which was processed with perfluoroethylene bromide (BrCF₂CF₂Br, Fluobrene®) at the temperature of 500° C. This material contains 1.84 mmol/g of bromine and 1.32 mmol/g fluorine and have a S_{BET}=905 m²/g. Instead of sodium hydrosulfide was used sodium mercaptoacetate concentrated solution (HSCH₂COONa), synthesis was performed at 120° C. during 12 h, and hydrochloric acid hydrolysis stage was performed by autoclaving S-adduct at 120° C. for 2 hours to hydrolyze grafted mercaptoacetic acid to acidum lacticum and surface thiol. After hydrolysis, product was oxidized by treatment of 3 g NaNO₂ in 3M nitric acid solution during 6 h. Based on the results of chemical analysis the resulting product contains 0.93 mmol/g of sulfur, 1.20 mmol/g of fluorine and not containing significant amounts of bromine. Resulting product have a S_{BET}=855 m²/g The results of TPD-mass, SO₂ desorption and catalytic studies show properties close to the product of Example 1.
3. 3 g of fluorinated carbon KAU (obtained by fruit stones carbonization and activation, S_{BET}=1100 m²/g) modified with Arcton® 3 (Freon R-13,CF₃Cl) at 550° C., that contain 1.06 mmol/g of fluorine and 0.88 mmol/g of chlorine, BET surface 752 m²/g, was treated in a stream of mixture argon and hydrogen sulfide (50 ml/min of argon, 2 ml/min of hydrogen sulfide) at 450° C. for 3 h. Subsequently, the resulting product was cooled in the reaction gas mixture and treated oxidation mixture and further as described in Example 1. The resulting fluorine-containing contains 1.81 mmol/ g of fluorine, 0.85 mmol/g of chlorine and 0.25 mmol/g of sulfur and has BET surface 680 m²/g (FIG. 7). The results TPD-mass studies sulfur desorption occurs in close to the product of Example 1 temperature range, as well as SO₂.
4. 1 g of multilayer carbon nanotubes (BET specific surface area of 180 m²/g), which was treated with R-134a, Forane® (H₂FC-CF₃) at 550° C., which contains fluorine in an amount of 0.25 mmol/g, were treated 5 ml 25% of oleum (25% SO₃ solution in H₂SO₄) at 60° C. After the reaction precipitate was filtered, washed with concentrated sulfuric acid, water, soda solution, 5% hydrochloric acid and again with water to neutral pH. The content of fluorine and sulfur in the product was 0.22 and 0.08 mmol/g, respectively.
5. 1 g of activated carbon fibers (raw material cellulose, BET surface area above 800 m²/g) which was treated with the refrigerant R-125, pentafluoroethane, at a temperature of 600° C., fluorine content which is 1.45 mmol/g, was treated with 2 g of chlorosulfonic acid, HSO₃Cl, at 60° C. for 3 hours, then treated with a product similar to Example 4. The resulting sulfurized product contains 1.30 mmol/g of fluoride, 0.27 mmol/g of sulfur and 0.03 mmol/g of chlorine.
6. 1 g of carbon black “K-354” that was processed with 2,2,2-trifluoroethanol (CF₃CH₂OH, 99+%, Alfa Aesar®) at a temperature of 660° C. (fluorine content is 1.05 mmol/g) was mixed with 5 g of thiourea and placed in a Pyrex® vial with thin exit. Ampoule was carefully heated to a temperature of 250° C. Moreover, there was a decomposition of sulfur-containing reagent to form gaseous products: CS₂, H₂NCN, ammonia and HNCS. After the evolution of gaseous products from reaction medium temperature was increased to 350° C. and vial evacuated was evacuated. After, the residue was washed with isopropanol, water, and oxidized similar to Example 1. The resulting sulfurized material contains 0.98 mmol/g of fluorine and 0.12 mmol/g of sulfur. According to TPD-mass research, evolving of sulfur dioxide observed in a temperature range of 130-450° C.
7. 3 g sample of fluorinated carbon material obtained by reacting of activated carbon Norit® 830W with R-134a at 600° C. (fluorine content is 1.93 mmol/g) in Teflon® vial was mixed with a mixture of sodium polysulfides hydrates and autoclaved at 175° C. for 12 hours. The resulting product was subjected to oxidation with 20% hydrogen peroxide in 50% acetic acid for 18 hours and further processed as described in Example 1. The resulting sulfurized product contains 1.85 mmol/g of fluoride and 0.44 mmol/g of sulfur. According to TPD-mass, only one product of sulfur desorption, SO₂, was observed.
8. A sample of 1.25 g of BAU-A activated carbon (raw material is the birch wood), that was treated with tetrafluoroethane, R-134a, Forane® (H₂FC-CF₃), at 500° C., which contains 0.42 mmol/g of fluorine and has a BET surface area about 550 m²/g was put in contact with argon, saturated with a steam of bis-tert-butyl disulfide (Di-tent-butyl disulfide, (CH₃)₃CSSC(CH₃)₃, Sigma-Aldrich®, 97%). Reactant gas mixture was obtained by bubbling of argon through a bis-tert-butyl disulfide at room temperature. Steam treatment was continued for 40 minutes at 475° C., and then passed through a sample of pure argon for 40 minutes and then the resulting sample was oxidized and processed similar to Example 1. The resulting sulfurized material contains 0.35 mmol/g of fluorine and 0.85 mmol/g of sulfur.
9. 1 g sample of fluorinated carbon material obtained by reacting of activated carbon Norit® 830W with R-125 at 600° C. (fluorine content is 2.30 mmol/g) was impregnated with 2.5 ml of a saturated solution of elemental sulfur in benzene, dried at 120° C. and placed in a glass vial. Vial was evacuated, sealed and heated to 465° C. for 2 hours. The resulting product is treated with 20% hydrogen peroxide acetic acid solution for 24 hours and then washed similar to Example 1. The resulting material contains 1.92 mmol/g of fluorine and 0.97 mmol/g of sulfur. According TG-DTG / TPD-IR data (Ar, 50 ml/min flow rate, 10 K./min heating rate, 60 mg sample, 100 mm long IR-cuvette with NaCl windows), the sample desorb sulfur in SO₂ form at mediate high temperature range (FIG. 8). For comparison, industrial sulfocoal “CK-1” that was washed by sodium carbonate to remove physisorbed H₂SO₄, after HCl 5% solution, to renew H-form of sulfonic groups, and by water again, to pH 4.5, was tested by similarly method. It was shown, that SO₂ desorbs from sample in the temperature of the range 290-590° C. (260-450° C. for CK-1) and contain significantly less (in comparison with sulfonated coal CK-1, CAS# 69013-20-3) of high temperature oxygen surface complexes, that desorbs in the temperature of the range 450-700° C. SO₂ evolving in this temperature range is agreed with TPD-IR data.

Evidently, numerous variations and modifications of the present invention are possible in light of the above studies. It is therefore to be understood that within the scope of the appended claims, the invention may be practiced otherwise than as specifically described in present Examples.

### CITATIONS

1. Nature Comm. 4, Art. NE 2162,—2013
2. Chem. Eng. J.—V. 315,—2017.—p. 598-607
3. WO 2014078846 A2, Pub. Date: May 22,2014
4. Phys. Chem. Chem. Phys. 2010.—12,—p. 8728-8732
5. U.S. Pat. No. 8,383,763 B2, Pub. Date: Feb. 26, 2013
6. U.S. Pat. No. 9,559,363 B2, Pub. Date: Jan. 31, 2017
7. U.S. Pat. No. 4,918,244 A, Pub. Date: Apr. 17, 1990
8. WO 2009032312 A1, Pub. Date: Mar. 12, 2009
9. U.S. Pat. No. 9,095,845 B2, Pub. Date: Aug. 4, 2015
10. WO 2012131718 A1 Pub. Date: Oct. 4, 2012
11. Cat. Comm.—Vol. 27,—2012.—p. 33-37
12. Theor. Exp. Chem.—Vol. 44(5),—2008.—p. 331-337
13. J. Phys. Chem. B.—2005.—109,—p. 17281-17289
14. Physica E. Low Dimens. Syst. Nanostruct.—41(4),—2009.—p. 723-728
15. Carbon,—Vol. 6,—1968.—p. 917-923
16. WO 2014082296 A1, Pub. Date: Jun. 5,2014
17. Carbon,—V. 45(4),—2007. p. 785-796

1. The method for preparing a functionalized carbon material with —CF₂SO₃H groups covalently bonded with the carbon matrix directly, or through a (per)fluoroalkylene chain, by chemical modification of a carbon material that contains fluoroorganic groups covalently bonded with the carbon matrix and capable of alkylating sulfur atoms with the formation of CF₂—S bonds [Material (FC)], said method comprising: contacting of said Material (FC) with a sulfur-containing compound that contain an atom of sulfur capable of alkylation [Reagent (S)], and heating of said Material (FC) while in contact with said Reagent (S) at a temperature sufficient to initiate the chemical reaction between said Material (FC) with said Reagent (S), wherein said atom of sulfur of the Reagent (S) is alkylated by said fluoroorganic groups of said Material (FC), and hydrolytic treatment of the obtained product of alkylation, and oxidation of the product of the hydrolytic treatment.

2. The method according to claim 1, wherein the Material (FC) is selected from the group consisting of the fluorinated activated carbon, fluorinated coke, fluorinated pitch coke, fluorinated charcoal, fluorinated carbon fibers, fluorinated carbon nanotubes, fluorinated carbon black, fluorinated graphene, fluorinated carbonizates, fluorinated nanodiamonds, fluorinated fullerenes or fluorinated fullerite.

3. The method according to claim 1, wherein the Reagent (S) is a substance selected from the group consisting of hydrogen sulfide, mercaptans, the salts of hydrogen sulphide, the salts of mercaptans, organic disulfide, sulfur oxide, sulfurous acid salts, thiourea, carbon disulfide, chlorosulfonic acid, polysulfide ion, and elemental sulfur.

4. The method according to claim 1, wherein said Material (FC) is obtained by chemical modification of a carbon material with the organofluorine compound, selected from the group consisting of fluorocarbons, halofluorocarbons, hydrofluorocarbons, or fluorinated alcohols.

5. The method according to claim 1, wherein the Material (FC) contains oxygen and/or hydrogen.

6. The method according to claim 1, wherein the Material (FC) contains double carbon-carbon bonds in the carbon matrix, capable of alkylating the sulfur atom of Reagent (S).

7. The method according to claim 1, wherein the interaction of Material (FC) with Reagent (S) is carried out in the liquid phase.

8. The method according to claim 1, wherein the interaction of Material (FC) with Reagent (S) is carried out in the gas phase.

9. The method according to claim 1, wherein the oxidation of the product of the hydrolytic treatment is carried out with a substance selected from the group consisting of hydrogen peroxide, nitric acid, nitrous acid, oxygen-halogen compounds, or molecular oxygen.
